(19) United States
(12) Patent Application Publication
(10) Pub. No.: US 2019/0269340 A1
(43) Pub. Date: Sep. 5, 2019

(54) BAG-SHAPED STRUCTURE AND MANUFACTURING METHOD THEREOF, CUFF, AND BLOOD PRESSURE MONITOR

(71) Applicants: OMRON CORPORATION, Kyoto-shi, Kyoto (JP); OMRON HEALTHCARE CO., LTD., Muko-shi, Kyoto (JP)

(72) Inventor: Kazuyoshi NISHIKAWA, Ritto-shi Shiga (JP)

(21) Appl. No.: 16/348,976

(22) PCT Filed: Dec. 26, 2017
(86) PCT No.: PCT/JP2017/046644

§ 371 Date: May 10, 2019

(30) Foreign Application Priority Data
Dec. 27, 2016 (JP) 2016-253788

(51) Int. Cl. A61B 5/022; A61B 5/00; C08J 5/18

(52) U.S. Cl. CPC A61B 5/02233; C08J 2375/04; C08J 5/18; A61B 5/681

(57) ABSTRACT

An improvement in performance of a bag-shaped structure formed of an elastomer-made sheet can be achieved. Provided is a bag-shaped structure including one or more sheets that contain a thermoplastic elastomer and are uniform in composition, wherein the one or more sheets comprise a plurality of regions that are different in 100% Modulus. Provided also is a method of manufacturing a bag-shaped structure, including heating a region of one or more sheets that contain a thermoplastic elastomer and are uniform in composition, to a temperature that is equal to or higher than a crystallization temperature of the thermoplastic elastomer and is lower than a melting point of the thermoplastic elastomer to increase a 100% Modulus at the region relative to another region.

### FIELD

The present invention relates to a bag-shaped structure, a method for manufacturing the same, a cuff, and a blood pressure monitor.

### BACKGROUND

Blood pressure measurement uses a cuff including a bag-shaped structure for the purpose of pressing blood vessels. This bag-shaped structure is formed of elastomer-made sheets so as to achieve good blood-vessel pressing characteristics when air is supplied to inflate the bag-shaped structure.

Jpn. Pat. Appln. KOKAI Publication No. 10-168305 discloses that a clay composite rubber material, which is obtained by swelling an organically modified layered clay mineral with an oligomer containing a hydrogen-bonding functional group capable of forming a hydrogen bond with the layered clay mineral, and by thereafter kneading the swollen layered clay mineral with a matrix such as rubber, has room for improvement in a gas barrier property and tensile strength. As a solution to this problem, Jpn. Pat. Appln. KOKAI Publication No. 10-168305 proposes a method of manufacturing a polyurethane composite material.

This method prepares an oligomer including a hydrogen-bonding functional group capable of forming a hydrogen bond with a layered clay mineral, and a urethane-bonding functional group capable of forming a urethane bond with an isocyanate compound. This oligomer is then mixed with an organically modified clay obtained by organic onium ion treatment of a layered clay mineral, thereby forming an oligomer-swollen organically modified clay. Thereafter, this is kneaded with an isocyanate compound to cause an urethanization reaction between the oligomer and the isocyanate compound.

### SUMMARY OF THE INVENTION

The inventor of the present invention has found that a bag-shaped structure formed of an elastomer-made sheet has room for improvement in terms of performance.

Accordingly, it is an object of the present invention to achieve an improvement in performance of a bag-shaped structure formed of an elastomer-made sheet.

According to a first aspect of the present invention, there is provided a bag-shaped structure comprising one or more sheets that contain a thermoplastic elastomer and are uniform in composition, wherein the one or more sheets comprise a plurality of regions that are different in 100% Modulus. Herein, “100% Modulus” refers to a value specified in “stress at a given elongation” in JIS K 6251:2010 (“Rubber, vulcanized or thermoplastic—Determination of tensile stress-strain properties”), that is, a tensile strength at 100% elongation. The test pieces used in this measurement are “type 3 dumb-bells” specified in JIS 6251:2010.

According to a second aspect of the present invention, there is provided a bag-shaped structure according to the first aspect, wherein the plurality of regions comprise a first region and a second region that is thinner and is higher in 100% Modulus than the first region.

According to a third aspect of the present invention, there is provided a bag-shaped structure according to the first or second aspect, wherein the one or more sheets are provided with one or more openings, the one or more sheets comprise a third region spaced apart from the one or more openings and a fourth region interposed between the one or more openings and the third region, and the fourth region has a 100% Modulus higher than that of the third region.

According to a fourth aspect of the present invention, there is provided a bag-shaped structure according to any one of the first to third aspects, wherein the one or more sheets are provided with a plurality of bonded regions which are bonded to each other, and the one or more sheets comprise a fifth region comprising the plurality of bonded regions and a sixth region, at least part of the fifth region having a 100% Modulus higher than that of the sixth region.

According to a fifth aspect of the present invention, there is provided a bag-shaped structure according to any one of the first to fourth aspects, wherein the one or more sheets comprise a seventh region, an eighth region that faces the seventh region in a case where the seventh region is applied on a living body, and a ninth region that connects an end of the seventh region and an end of the eighth region together, at least part of the ninth region having a 100% Modulus of higher than that of the seventh region. The case in which the seventh region is applied on a living body includes not only the case in which the seventh region is brought in direct contact with a living body but also the case in which the seventh region is brought in indirect contact with a living body via another substance.

According to a sixth aspect of the present invention, there is provided a bag-shaped structure according to the fifth aspect, wherein the ninth region includes a bonded region at which the one or more sheets are bonded to each other, and comprises a region whose 100% Modulus is higher than the seventh region between the bonded region of the ninth region and the seventh region.

According to a seventh aspect of the present invention, there is provided a cuff for a blood pressure monitor, comprising the bag-shaped structure according to any one of the first to sixth aspects.

According to an eighth aspect of the present invention, there is provided a blood pressure monitor comprising the cuff according to the seventh aspect.

According to a ninth aspect of the present invention, there is provided a method of manufacturing a bag-shaped structure, comprising heating a region of one or more sheets that contain a thermoplastic elastomer and are uniform in composition, to a temperature that is equal to or higher than a crystallization temperature of the thermoplastic elastomer and is lower than a melting point of the thermoplastic elastomer to increase a 100% Modulus at the region relative to another region.

According to a tenth aspect of the present invention, there is provided a method of manufacturing a bag-shaped structure according to the ninth aspect, wherein the one or more sheets further contain a nucleating agent.

According to the first aspect, one or more sheets that contain a thermoplastic elastomer and are uniform in composition are provided with a plurality of regions that are different in 100% Modulus. Thus, the bag-shaped structure can be improved in performance. For example, good flexibility can be achieved in a region having a lower 100% Modulus while high strength can be achieved in a region having a higher 100% Modulus.

According to the second aspect, the plurality of regions comprise a first region and a second region that is thinner and is higher in 100% Modulus than the first region. Thus, for example, insufficient strength due to a small thickness can be prevented.

According to the third aspect, one or more sheets are provided with one or more openings, and in the one or more sheets, as compared to a 100% Modulus of a third region spaced apart from the one or more openings, a 100% Modulus of a fourth region interposed between the one or more openings and the third region is made higher. Thus, for example, insufficient strength in the regions near the openings can be prevented.

According to the fourth aspect, one or more sheets are provided with a plurality of bonded regions which are bonded to each other, the one or more sheets comprise a fifth region comprising the plurality of bonded regions and a sixth region, and a 100% Modulus of at least part of the fifth region is higher than the sixth region. Thus, insufficient strength can be prevented in, for example, the bonded regions or their vicinities.

According to the fifth aspect, one or more sheets include a seventh region, an eighth region that faces the seventh region when the seventh region is applied on a living body, and a ninth region that connects an end of the seventh region and an end of the eighth region together, and a 100% Modulus of at least part of the ninth region is higher than the seventh region. Thus, for example, while the seventh region that presses a living body is provided with sufficient flexibility, abnormal inflation of the bag-shaped structure can be suppressed.

According to the sixth aspect, the ninth region includes a bonded region at which one or more sheets are bonded to each other, and comprises a region whose 100% Modulus is higher than the seventh region between the bonded region of the ninth region and the seventh region. Thus, for example, while flexibility in the seventh region that presses a living body is maintained, insufficient strength can be prevented in the region (the region between the bonded region and the seventh region) in which the pressure is concentrated most when pressing a living body, and abnormal inflation of the bag-shaped structure can be suppressed.

According to the seventh aspect, the bag-shaped structure according to any one of the first to sixth aspects is used in a cuff for a blood pressure monitor. Thus, superior artery occlusion characteristics can be attained.

According to the eighth aspect, the cuff according to the seventh aspect is used in a blood pressure monitor. Thus, a blood pressure value can be measured with high accuracy.

According to the ninth aspect, a region of one or more sheets that contain a thermoplastic elastomer and are uniform in composition is heated to a temperature that is equal to or higher than a crystallization temperature of the thermoplastic elastomer and is lower than a melting point of the thermoplastic elastomer to increase a 100% Modulus at the region relative to another region. Thus, for example, the bag-shaped structure can be improved in performance by a simple method. For example, good flexibility can be achieved in a region having a lower 100% Modulus while high strength can be achieved in a region having a higher 100% Modulus.

According to the tenth aspect, one of more sheets further contain a nucleating agent. Thus, the sheets can be easily varied in 100% Modulus.

### BRIEF DESCRIPTION OF THE DRAWINGS

FIG. 1 is a perspective view schematically showing a blood pressure monitor according to one embodiment of the present invention;

FIG. 2 is a cross-sectional view taken along line II-II of the blood pressure monitor in FIG. 1;

FIG. 3 is a broken perspective view of a bag-shaped structure included in the blood pressure monitor shown in FIGS. 1 and 2;

FIG. 4 is a cross-sectional view of the bag-shaped structure included in the blood pressure monitor shown in FIGS. 1 and 2;

FIG. 5 is a cross-sectional view schematically showing an example of a plurality of regions that are different in 100% Modulus;

FIG. 6 is a perspective view schematically showing another example of a plurality of regions that are different in 100% Modulus;

FIG. 7 is a cross-sectional view schematically showing a state in which a cuff included in the blood pressure monitor shown in FIGS. 1 and 2 is fitted on a living body;

FIG. 8 is a cross-sectional view schematically showing the same state as in FIG. 7 except that the bag-shaped structure included in the cuff is inflated;

FIG. 9 is a cross-sectional view schematically showing a state in which a bag-shaped structure according to an example is inflated in the same manner as in FIG. 8;

FIG. 10 is a cross-sectional view schematically showing a state in which a bag-shaped structure according to another example is inflated in the same manner as in FIG. 8;

FIG. 11 is a perspective view schematically showing a state in which abnormal inflation occurs in a bag-shaped structure; and

FIG. 12 is a perspective view schematically showing a blood pressure monitor according to another embodiment of the present invention.

### DETAILED DESCRIPTION

Hereinafter, embodiments of the present invention will be described with reference to the drawings. Elements having the same or similar functions are denoted by the same reference numerals, and redundant explanations will be omitted.

<Blood Pressure Monitor>

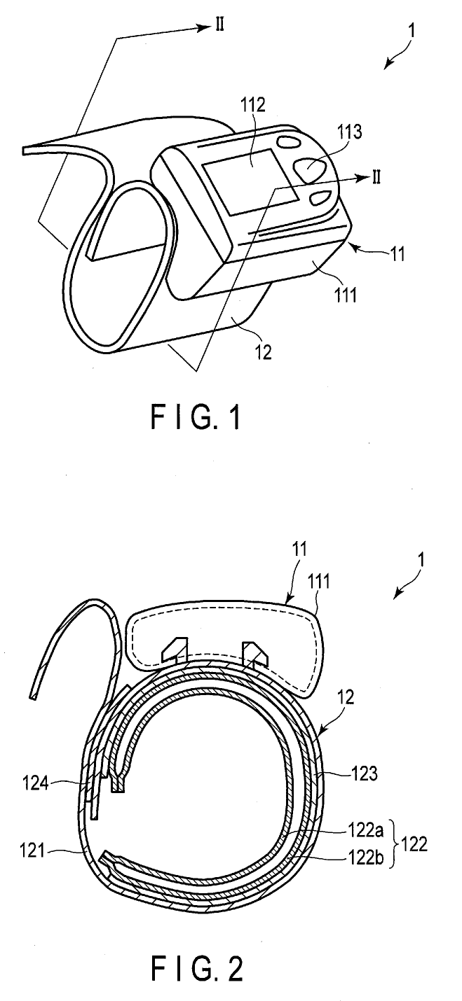

FIG. 1 is a perspective view schematically showing a blood pressure monitor according to one embodiment of the present invention. FIG. 2 is a cross-sectional view taken along line II-II of the blood pressure monitor in FIG. 1.

The blood pressure monitor 1 shown in FIG. 1 is an electronic wrist blood pressure monitor. The blood pressure monitor 1 includes an apparatus body 11 and a cuff 12.

The apparatus body 11 includes a housing 111, a display unit 112, and an operation unit 113. The apparatus body 11 further includes a flow path, a pump, a valve, a pressure sensor, a control unit, and a power supply unit (none of which are shown).

The upper part of the housing 111 includes openings for the display unit 112 and the operation unit 113. Furthermore, the lower part of the housing 111 is provided with a configuration for detachably fixing the apparatus body 11 to the cuff 12, in this case, recesses in which claws provided in the cuff 12 are inserted.

The display unit 112 is installed in the housing 111 so as to display images at the position of the opening provided in the upper part of the housing 111. The display unit 112 is, for example, a liquid crystal display or an organic electroluminescence display. The display unit 112 displays various types of information including a measurement result such as blood-pressure values of, e.g., the highest and lowest blood pressures, a heart rate, etc.

The operation unit 113 includes buttons for a user to, e.g., start/stop measurement, turn on/off the power supply, select functions, and make various settings. The operation unit 113 is installed in the housing 111 so that those buttons are exposed to the external space of the housing 111 at the position of the opening described above. The operation unit 113 outputs electric signals corresponding to commands or information input via the buttons.

According to an example, the flow path has a structure branched in four directions and includes four openings. One of those openings is connected to a supply/exhaust port of the bag-shaped structure 122 included in the cuff 12.

The pump is installed in the housing 111. An exhaust port of the pump is connected to another one of the openings included in the flow path. The pump is, for example, a rolling pump. The pump discharges compressed air from its exhaust port.

The valve is installed in the housing 111. The valve is connected to yet another one of the openings included in the flow path. The valve is a valve whose operation is controllable using electric power, for example, a solenoid valve. The valve opens and closes the opening to which the valve is attached.

The pressure sensor is installed in the housing 111. The pressure sensor is connected to the remaining one of the openings included in the flow path. The pressure sensor is, for example, a piezoresistive type pressure sensor. The pressure sensor detects pressure within the flow path and outputs an electric signal corresponding to this pressure.

The control unit is installed in the housing 111. The control unit is electrically connected to the display unit 112, the operation unit 113, the pump, the valve, and the pressure sensor, and supplies electric power to them. Furthermore, the control unit controls the operation of the display unit 112, the pump, and the valve based on electric signals output from the operation unit 113 and the pressure sensor. For example, when an electric signal corresponding to the start of measurement is supplied from the operation unit 113, the control unit controls the operation of the valve and the pump in a manner so that the valve is closed and then the pump starts driving. The control unit then determines a timing for stopping the operation of the pump, based on electric signals output from the pressure sensor, and controls the operation of the pump and the valve in a manner so that the pump stops operating at the determined timing and then the valve is gradually opened. Subsequently, the control unit obtains a measurement result such as blood-pressure values of the highest and lowest blood pressures, and a heart rate, from electric signals output from the pressure sensor, and outputs video signals corresponding to the measurement result to the display unit 112.

The power supply unit is installed in the housing 111. The power supply unit includes a battery. The power supply unit supplies electric power to the control unit.

The cuff 12 is detachably attached to the apparatus body 11. The cuff 12 is wrapped around a living body, specifically, a wrist of the living body and inflates in this state so as to press an artery.

As shown in FIG. 2, the cuff 12 includes a cover member 121, a bag-shaped structure 122, a curler 123, and a fastener 124.

The cover member 121 is installed in a manner to face a living body with the bag-shaped structure 122 interposed therebetween when the cuff 12 is fitted on the living body. The cover member 121 is a sheet having poor stretchability. The cover member 121 forms a structure having a bag shape extending in one direction. This direction corresponds to a direction in which the cuff 12 is wrapped around when the cuff 12 is fitted on a living body.

As will be described later, the cover member 121 supports the bag-shaped structure 122 and the curler 123, and enables the cuff 12 to be wrapped around a living body. Furthermore, when the bag-shaped structure 122 is inflated, the cover member 121 suppresses inflation toward the side opposite to a living body without hindering inflation toward the living body.

The bag-shaped structure 122 is supported by the cover member 121. As described above, the bag-shaped structure 122 includes the supply/exhaust port, and this supply/exhaust port is connected to one of the openings of the flow path included in the apparatus body 11. The bag-shaped structure 122 may include a supply port and an exhaust port instead of the supply/exhaust port.

When the pump is driven with the cuff 12 being fitted on a living body and the valve being closed, the bag-shaped structure 122 inflates and as a result, the cuff 12 occludes an artery of the living body. Subsequently, when the driving of the pump is stopped and the valve is opened, the bag-shaped structure 122 deflates and as a result, the pressure applied to the living body by the cuff 12 decreases, and the flow of blood is resumed. Details of the bag-shaped structure 122 will be described later.

The curler 123 is positioned between the cover member 121 and the bag-shaped structure 122. The curler 123 is fixed to the cover member 121 and the bag-shaped structure 122 by adhesion means such as a double-sided tape. The curler 123 is an elastic member made of, for example, a resin such as polypropylene. Furthermore, the curler 123 has a shape curved in its longitudinal direction. This enables the curler 123 to curve the cuff 12 in its longitudinal direction, thereby facilitating the attachment of the cuff 12 to a living body. The curler 123 can be omitted.

The fastener 124 functions to fix one end of the cover member 121 to the other end when the cuff 12 is fitted on a living body. The fastener 124 is, for example, a hook-and-loop fastener. In this case, a hook surface of the hook-and-loop fastener is provided in one of regions that are paired with each other and are positioned on surfaces of the cover member 121 in a manner to face each other when the cuff 12 is fitted on a living body, whereas a loop surface of the hook-and-loop fastener is provided in the other one of the regions.

<Bag-Shaped Structure>

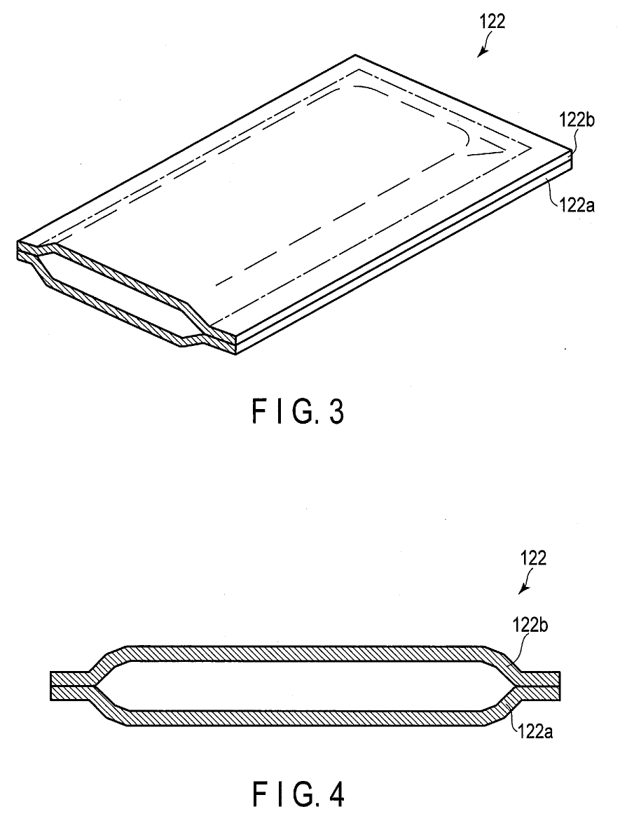

Next, the bag-shaped structure 122 will be described in detail with reference to FIGS. 3 and 4.

FIG. 3 is a broken perspective view of the bag-shaped structure included in the blood pressure monitor shown in FIGS. 1 and 2. FIG. 4 is a cross-sectional view of the bag-shaped structure included in the blood pressure monitor shown in FIGS. 1 and 2.

The bag-shaped structure 122 shown in FIGS. 3 and 4 includes one or more sheets, herein, sheets 122a and 122b.

The sheets 122a and 122b are bonded to each other at their peripheral portions. This bonding can be performed by laser welding, high frequency welding, hot press welding, or adhesion with an adhesive or a double-sided tape.

The sheets 122a and 122b contain a thermoplastic elastomer.

As the thermoplastic elastomer, for example, polyurethane resin (TPU), vinyl chloride resin (PVC), ethylene-vinyl acetate resin (EVA), polystyrene resin (TPS), polyolefin resin (TPO), polyester resin (TPEE), and polyamide resin (TPA) can be used. As the thermoplastic elastomer, it is preferable to use a thermoplastic polyurethane resin.

A nucleating agent may be added to the thermoplastic elastomer. When the nucleating agent is added to the thermoplastic elastomer, a distribution in 100% Modulus described later is easily caused. As the nucleating agent, for example, compounds such as talc, clay, metal salt of organic acid, and metal oxide can be used. The amount of nucleating agent is preferably 3.0 parts by mass or less, more preferably in the range from 0.1 parts by mass to 1.0 part by mass, with respect to 100 parts by mass of thermoplastic elastomer.

In the case of adopting the configuration in which the bag-shaped structure 122 comes in contact with a skin, an additive such as silica, calcium carbonate, or talc may be added to the thermoplastic elastomer. Usage of such an additive improves the sheet in terms of skin sensation. The amount of this additive is preferably 10 parts by mass or less, more preferably in the range from 0.5 parts by mass to 2.0 parts by mass, with respect to 100 parts by mass of thermoplastic elastomer.

At least one of the sheets 122a and 122b is uniform in composition. The sheets 122a and 122b may be the same or different in composition. As an example, herein, it is assumed that the sheets 122a and 122b are each uniform in composition and are the same in composition.

Although the sheets 122a and 122b are the same in composition, they contain a plurality of regions that are different in 100% Modulus. A region with a higher 100% Modulus is higher in strength than a region with a lower 100% Modulus. A region with a lower 100% Modulus is superior in flexibility to a region with a higher 100% Modulus.

Herein, “100% Modulus” refers to a value specified in “stress at a given elongation” in JIS K 6251:2010 (“Rubber, vulcanized or thermoplastic—Determination of tensile stress-strain properties”). The test pieces used in this measurement are “type 3 dumb-bells” specified in JIS 6251:2010.

A region with the highest 100% Modulus has a 100% Modulus of preferably 5.0 MPa or less, more preferably, 3.0 MPa or less. A region with the lowest 100% Modulus has a 100% Modulus of preferably 0.1 MPa or more, more preferably, 0.5 MPa or more. A difference in 100% Modulus between a region with the highest 100% Modulus and a region with the lowest 100% Modulus is preferably in the range from 0.3 MPa to 3.0 MPa, more preferably the range from 0.5 MPa to 1.0 MPa. If a 100% Modulus is too high, flexibility may be insufficient. If a 100% Modulus is too low, strength may be insufficient. If a difference in 100% Modulus is too small, a difference in flexibility and strength does not stand out. If a difference in 100% Modulus is excessively large, there is an increased possibility that a region with a too high or too small 100% Modulus is present.

A 100% Modulus can be adjusted according to the type of compound used for a thermoplastic elastomer, the ratio of a hard segment content to a soft segment content contained therein, the presence or absence of an additive, the type and content of an additive, and conditions of heat treatment described later.

According to an example, the plurality of regions that are different in 100% Modulus include a first region and a second region that is thinner and is higher in 100% Modulus than the first region.

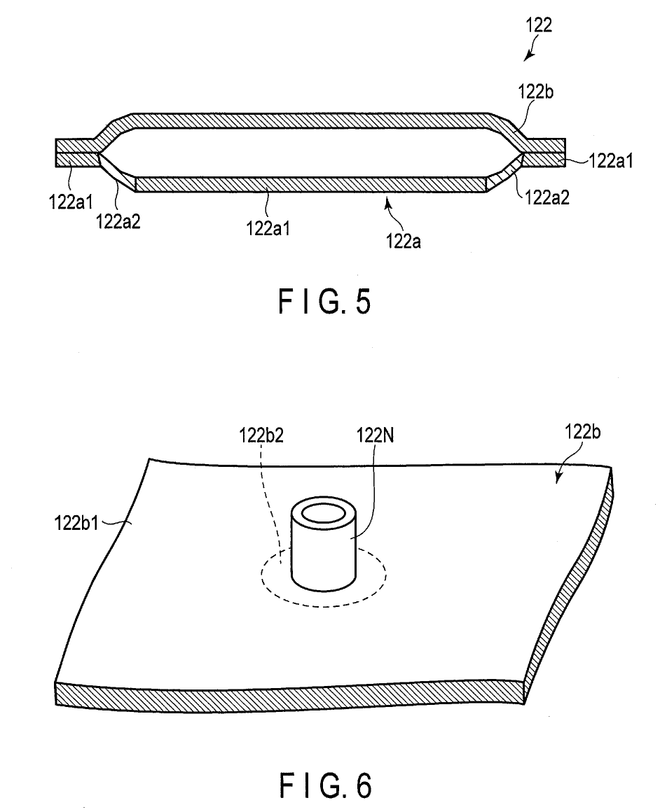

FIG. 5 is a cross-sectional view schematically showing an example of a plurality of regions that are different in 100% Modulus. In the bag-shaped structure 122 shown in FIG. 5, the sheet 122a includes regions 122a1 and 122a2. The region 122a2 is thinner and has a higher 100% Modulus than the region 122a1. That is, in the configuration shown in FIG. 5, the region 122a1 corresponds to the first region while the region 122a2 corresponds to the second region.

According to another example, one or more sheets are provided with one or more openings, and include a third region spaced apart from the one or more openings and a fourth region interposed between the one or more openings and the third region, in which a 100% Modulus of the fourth region is higher than that of the third region.

FIG. 6 is a perspective view schematically showing another example of a plurality of regions that are different in 100% Modulus. In the configuration shown in FIG. 6, an opening is provided in a sheet 122b, and a nipple 122N forming a supply/exhaust port is attached to this opening. The sheet 122b includes a region 122b1 spaced apart from the aforementioned opening and a region 122b2 interposed between the opening and the region 122b1. The region 122b2 has a higher 100% Modulus than the region 122b1. That is, in this configuration, the region 122b1 corresponds to the third region while the region 122b2 corresponds to the fourth region.

According to yet another example, one or more sheets are provided with a plurality of bonded regions which are bonded to each other, and the one or more sheets include a fifth region including the plurality of bonded regions and a sixth region, in which a 100% Modulus of at least part of the fifth region is higher than that of the sixth region.

According to still another example, one or more sheets include a seventh region, an eighth region that faces the seventh region in the case where the seventh region is applied on a living body, and a ninth region that connects an end of the seventh region and an end of the eighth region together, in which a 100% Modulus of at least part of the ninth region is higher than that of the seventh region.

In other words, one or more sheets forming the bag-shaped structure 122 include a first portion, a second portion, and a third portion, in which the first portion is positioned between an internal space of the bag-shaped structure 122 and a living body when the cuff 12 is fitted on a living body, the second portion faces the first portion with the aforementioned internal space interposed therebetween when the cuff 12 is fitted on the living body, and the third portion connects an end of the first portion and an end of the second portion together. At least part of the third portion has a higher 100% Modulus than the first portion.

Preferably, the ninth region includes a bonded region at which one or more sheets are bonded to each other, and a region whose 100% Modulus is higher than the seventh region between the bonded region of the ninth region and the seventh region.

In the configuration shown in FIG. 5, the seventh region corresponds to a portion of the region 122a1 that is sandwiched between the regions 122a2, and the eighth region corresponds to a region of the sheet 122b that faces the seventh region. The ninth region corresponds to regions at both ends of the sheets 122a and 122b. The bonded region corresponds to regions of the sheets 122a and 122b that are bonded to each other at their both ends. In this configuration, within the ninth region, the regions 122a2 positioned between the bonded region and the seventh region have a higher 100% Modulus than the seventh region.

Note that two or more of the configurations described above may be combined with each other.

A region with a higher 100% Modulus has higher crystallinity of thermoplastic elastomer than a region with a lower 100% Modulus. As will be described later, a distribution of this crystallinity can be generated by performing a predetermined heat treatment.

Regarding the thickness of sheets forming the bag-shaped structure 122, in the examples shown in FIGS. 3 and 4, the thickness of the sheets 122a and 122b is preferably in the range from 0.03 mm to 0.60 mm, more preferably in the range from 0.10 mm to 0.40 mm. If this thickness is too small, the effect of improving strength by increasing crystallinity is small. If this thickness is too large, there is a possibility that the low thermal conductivity of thermoplastic elastomer causes a large difference in degree of crystallinity between a portion in which sheets overlap each other and a portion in which sheets do not overlap.

<Manufacture of Bag-Shaped Structure>

The bag-shaped structure 122 described above is manufactured, for example, by the following method.

First, a sheet containing a thermoplastic elastomer is prepared. Then, this sheet is cut to obtain the sheets 122a and 122b. A nucleating agent or another additive may be added to the thermoplastic elastomer.

Next, a predetermined heat treatment is applied to a region of at least one of the sheets 122a and 122b, specifically, a region whose 100% Modulus is desired to be made higher than those of another region. That is, a region of at least one of the sheets 122a and 122b is heated to a temperature that is equal to or higher than the crystallization temperature of the thermoplastic elastomer and is lower than the melting point of the thermoplastic elastomer to increase a 100% Modulus at this region relative to the other regions.

When the thermoplastic elastomer is heated to a temperature that is equal to or higher than its crystallization temperature and is lower than its melting point, the crystallinity or degree of crystallinity is increased. As a result, a 100% Modulus is increased. If a temperature of this heat process is lower than the crystallization temperature of the thermoplastic elastomer, rearrangement of the thermoplastic elastomer molecules does not occur, thus a 100% Modulus is not increased. In addition, when a temperature of this heat treatment is equal to the melting point of the thermoplastic elastomer or higher, a temperature tracks the same temperature history as at the time of manufacturing the sheets, so that a 100% Modulus is not increased in a similar way.

If this temperature is too low, a long time is required for the heat treatment. If this temperature is too high, there is a possibility that the temperature of the thermoplastic elastomer exceeds its melting point due to unintentional fluctuation in heat treatment temperature. In the case of using a thermoplastic polyurethane resin as the thermoplastic elastomer, this heating is preferably carried out in the range of 70° C. to 120° C.

In the case of using a thermoplastic polyurethane resin as the thermoplastic elastomer, the duration of this heat treatment is preferably in the range of 10 minutes to 1 hour. If this duration is too short, the degree of crystallinity of the thermoplastic elastomer does not increase. If this duration is too long, it is difficult to achieve high productivity.

This heat treatment is performed using one or more of, for example, a laser, an infrared heater, and a metal jig equipped with a heater.

For a heat treatment using a laser, for example, a semiconductor laser is used. According to an example, by repeatedly scanning a laser beam on the sheet in a line shape with the focal diameter of 2 mm, the sheet is heated at an irradiation position of a laser beam. The laser output and the scanning speed are appropriately set in a manner so that a temperature of an irradiated portion falls within the above range. In the case of using a thermoplastic polyurethane resin as the thermoplastic elastomer, the laser output is set in the range of, for example, 0.2 W to 5.0 W, and the scanning speed is set in the range of, for example, 3 mm/sec to 30 mm/sec.

Since heating with an infrared heater is radiation heating, it is preferable that a heat treatment is performed with a region that should not be heated, being cooled by applying cold air, for example. In this manner, a region to be heated can be prevented from broadening excessively.

Heating with a metal jig is performed, for example, in a manner so that a cartridge heater is attached to the metal jig and a portion to be heated of the sheet is nipped and heated with the metal jig.

This heat treatment is performed selectively on one region of the sheet, for example. In this case, a 100% Modulus of a region subjected to the heat treatment can be made higher than those of the other regions.

This heat treatment may be performed on a plurality of regions under different conditions. For example, the heat treatment may be performed on one region of the sheet under the first condition that falls within the above-described range, and be performed on another region of the sheet under the second condition that falls within the above-described range and is different from the first condition. In this case, 100% moduli increase in both one region and another one region but are different therebetween. For the difference between the first and second conditions, it is sufficient that they are different in at least one of a temperature and a duration time of a heat treatment.

After the heat treatment described above, the sheets 122a and 122b are bonded to each other at their peripheral portions. As described above, this bonding can be performed by laser welding, high frequency welding, hot press welding, or adhesion with an adhesive or a double-sided tape.

In this manner, the bag-shaped structure 122 is obtained.

In this method, the heat treatment for increasing a 100% Modulus is performed immediately before the sheets 122a and 122b are bonded together; however, this heat treatment may be performed before cutting the sheet into the sheets 122a and 122b. Alternatively, this heat treatment may be performed after the sheets 122a and 122b are bonded together.

<Measurement of Blood Pressure Value>

Next, the measurement of blood pressure values using the blood pressure monitor 1 will be described with reference to FIGS. 1, 2, and 7 to 10.

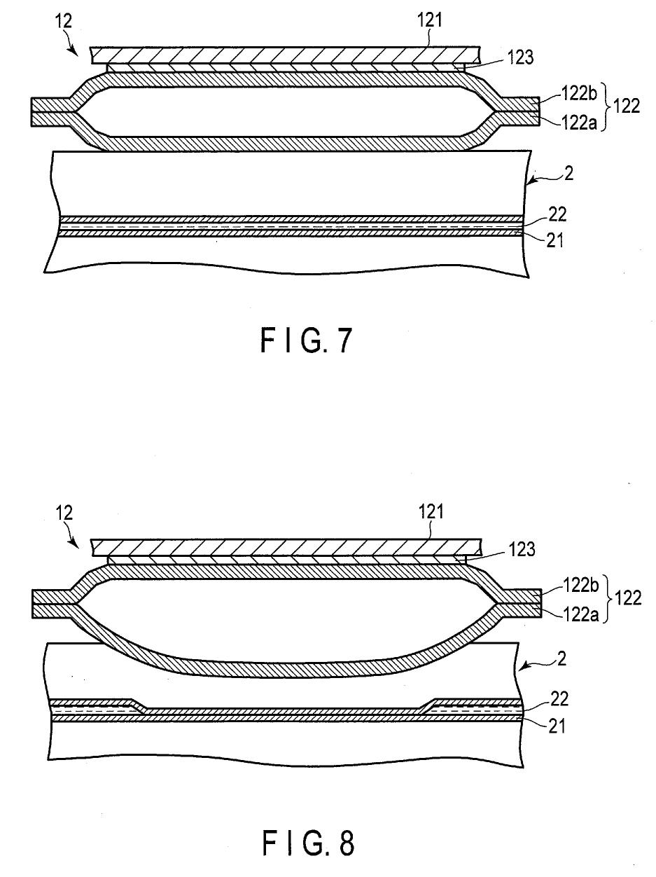

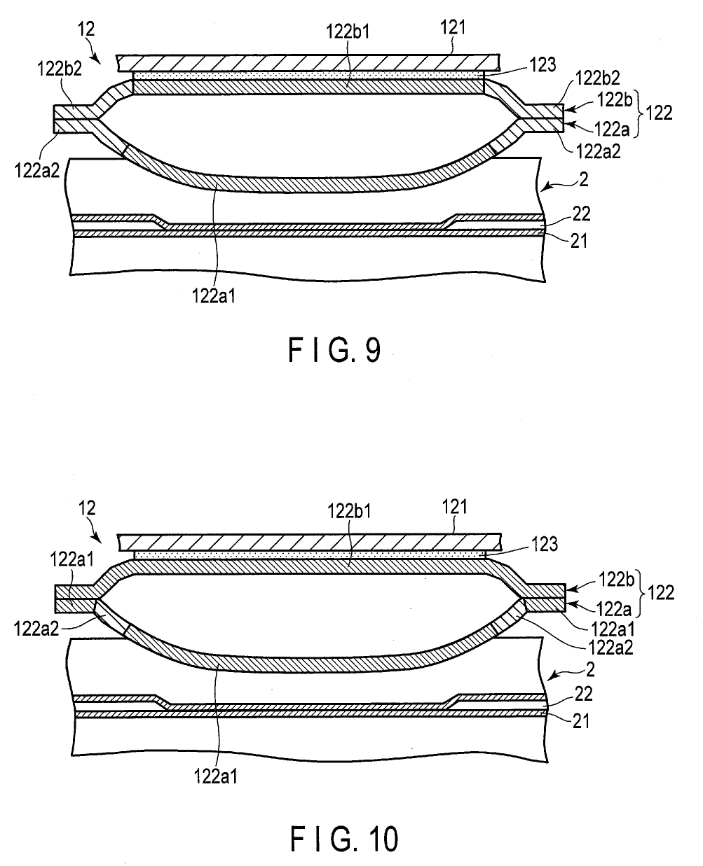

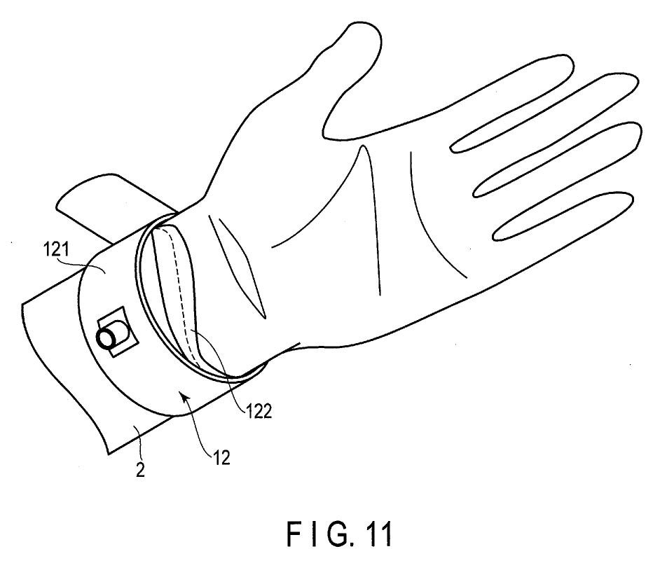

FIG. 7 is a cross-sectional view schematically showing a state in which the cuff included in the blood pressure monitor shown in FIGS. 1 and 2 is fitted on a living body. FIG. 8 is a cross-sectional view showing the same state as in FIG. 7 except that the bag-shaped structure included in the cuff shown in FIG. 7 is inflated. FIG. 9 is a cross-sectional view schematically showing a state in which the bag-shaped structure according to an example is inflated in the same manner as in FIG. 8. FIG. 10 is a cross-sectional view schematically showing a state in which the bag-shaped structure according to another example is inflated in the same manner as in FIG. 8. In the following description, a person to be measured performs all operations by himself or herself.

To measure a blood pressure value, a person to be measured first wears the cuff 12 on the wrist 2 as shown in FIG. 7. Next, the person to be measured operates the operation unit 113 shown in FIG. 1 to input a command corresponding to the start of measurement of a blood pressure value.

When this command is input, the operation unit 113 outputs an electric signal corresponding to the start of measurement to the control unit. The control unit supplied with this signal controls the operation of the valve and the pump in a manner so that the valve is closed and the pump starts driving. In this manner, the bag-shaped structure 122 starts inflating.

The pressure sensor detects a pressure in the internal space of the bag-shaped structure 122 and outputs an electric signal corresponding to this pressure to the control unit. Based on this electrical signal, the control unit determines whether or not the pressure in the internal space of the bag-shaped structure 122 has reached a predetermined level for blood pressure measurement. The control unit then controls the operation of the pump so that the pump halts driving when this pressure has reached the aforementioned level. Immediately after the pump halts driving, as shown in FIG. 8, the bag-shaped structure 122 is sufficiently inflated, and the cuff 12 occludes an artery 21 at the position of a wrist 2.

Here, as an example, assume that the bag-shaped structure 122 has the configuration shown in FIG. 9 or 10.

According to the configurations shown in FIGS. 9 and 10, the region 122a1 of the sheet 122a that is in contact with the wrist 2 has a 100% Modulus smaller than that of the other region 122a2. According to the configurations shown in FIGS. 9 and 10, the region of the sheet 122b that is in contact with the curler 123 also has a 100% Modulus smaller than that of the region 122a2. The region of the sheet 122b that is in contact with the curler 123 may have a 100% Modulus equal to that of the region 122a2.

According to the configurations shown in FIGS. 9 and 10, at least part of the region that connects the end of the region 122a1 of the sheet 122a that is in contact with the wrist 2, to the end of the region of the sheet 122b that is in contact with the curler 123, has a higher 100% Modulus than the region 122a1 of the sheet 122a that is in contact with the wrist 2. Preferably, a region that is interposed between the bonded regions of the sheets 122a and 122b that are bonded together and the region 122a1 of the sheet 122a that is in contact with the wrist 2 has a higher 100% Modulus than the region 122a1 of the sheet 122a that is in contact with the wrist 2. As will be described later, according to this configuration, a region in which the pressure is concentrated has a high 100% Modulus, so that abnormal inflation hardly occurs.

After the bag-shaped structure 122 is sufficiently inflated as described above, the control unit controls the operation of the valve so that the valve is gradually opened. When the valve is opened, the air inside the bag-shaped structure 122 is exhausted, thereby lowering the pressure in the internal space. In this decompression process, the flow of blood 22 in the artery 21 is resumed. From electric signals output from the pressure sensor in this process, the control unit obtains a measurement result such as blood-pressure values of, e.g., the highest and lowest blood pressures, a heart rate, etc., and outputs video signals corresponding to the measurement result to the display unit 112 shown in FIG. 1.

When the aforementioned video signals are supplied, the display unit 112 displays on its screen the measurement result such as blood-pressure values of, e.g., the highest and lowest blood pressures, a heart rate, etc. In this way, the measurement is terminated.

<Effect>

To achieve superior blood-vessel pressing characteristics, it is advantageous for the sheets forming the bag-shaped structure 122 to have high flexibility. However, if all the sheets have high flexibility, that is, if sheets not subjected to the heat treatment for increasing a 100% Modulus is used, the following problem may occur.

FIG. 11 is a perspective view schematically showing a state in which abnormal inflation occurs in a bag-shaped structure. FIG. 11 illustrates a state in which the blood pressure monitor 1 shown in FIGS. 1 and 2 is fitted on the wrist 2 and the bag-shaped structure 122 is inflated. In FIG. 11, the apparatus body 11 is omitted.

When the cuff 12 in which all the sheets forming the bag-shaped structure 122 have high flexibility is fitted on the wrist 2 and the bag-shaped structure 122 is inflated, there is a possibility that the bag-shaped structure 122 is inflated widely in the width direction, as shown in FIG. 11. That is, there is a possibility that the bag-shaped structure 122 causes abnormal inflation. Since the sheet 122b is adhered to the curler 123, the inflation in the width direction of the bag-shaped structure 122 tends to occur in a region interposed between the bonded region of the sheet 122a at which the sheets 122a and 122b are bonded to each other and a region of the sheet 122a that is in contact with the wrist 2.

When such abnormal inflation occurs, the pressure is not effectively applied to the wrist 2, thereby causing abnormality in a waveform of a pulse wave. As a result, it becomes difficult to measure a blood pressure value with accuracy.

The occurrence of abnormal inflation can be prevented by decreasing the flexibility of the sheets forming the bag-shaped structure 122. However, this case involves a possibility that the conformability of the cuff 12 to a living body at the time of inflation of the bag-shaped structure 122 is not sufficient to attain superior artery occlusion characteristics.

In the bag-shaped structure 122 described with reference to FIGS. 1 to 4, one or more sheets forming this bag-shaped structure 122 include a plurality of regions that are different in 100% Modulus. Therefore, for example, in one or more sheets forming the bag-shaped structure 122, a 100% Modulus of a region that tends to cause abnormal inflation can be made higher than a 100% Modulus of a region that comes in contact with a living body. Therefore, for example, in the case of adopting this configuration, even if the cuff 12 is decreased in width, the occurrence of abnormal inflation can be prevented without sacrificing the conformability of the cuff 12 to a living body at the time of inflating the bag-shaped structure 122. That is, in this case, even if the cuff 12 is decreased in width, a blood pressure value can be measured with accuracy.

In the bag-shaped structure 122 described with reference to FIGS. 1 to 4, of the sheets forming the bag-shaped structure 122, for example, a region having a small thickness, a region adjacent to the supply/exhaust port, or a bonded region and its neighboring region can have a 100% Modulus higher than a 100% Modulus of a region adjacent thereto.

Accordingly, even with the use of sheets of a single type, the bag-shaped structure 122 described with reference to FIGS. 1 to 4 can satisfy various performances required for a bag-shaped structure of the cuff 12, such as mechanical strength, a peeling resistance, etc. That is, the bag-shaped structure 122 described with reference to FIGS. 1 to 4 can achieve a high performance with a simple structure, thereby having a cost advantage, too.

As described above, according to the above technique, a bag-shaped structure formed of an elastomer-made sheet can be improved in performance.

<Another Application Example of Bag-Shaped Structure>

In the above, the blood pressure monitor 1 shown in FIGS. 1 and 2 was described as an application example of the bag-shaped structure 122. However, the bag-shaped structure 122 can also be used in other blood pressure monitors.

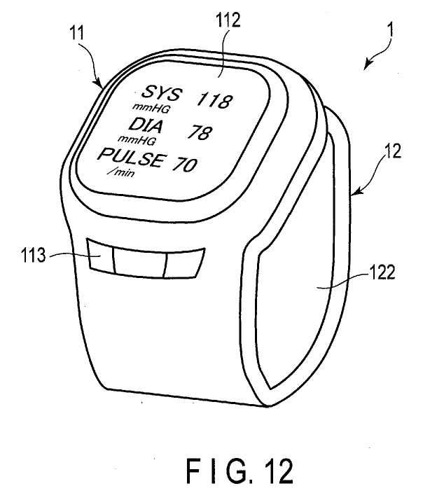

FIG. 12 is a perspective view schematically showing a blood pressure monitor according to another embodiment of the present invention.

The blood pressure monitor 1 shown in FIG. 12 is an electronic wrist blood pressure monitor of a wristwatch type. This blood pressure monitor 1 is smaller than the blood pressure monitor 1 described with reference to FIGS. 1 and 2. In this type of the blood pressure monitor 1, the apparatus body 11 and the cuff 12 are integrally formed. Other than these, the blood pressure monitor 1 shown in FIG. 12 has substantially the same structure as that of the blood pressure monitor 1 described with reference to FIGS. 1 and 2.

With the bag-shaped structure 122, for example, in the case where the cuff 12 is decreased in width to, for example, 40 mm or less, or even 20 mm or less, the occurrence of abnormal inflation can be prevented without sacrificing the conformability of the cuff 12 to a living body at the time of inflating the bag-shaped structure 122. That is, in this case, even if the cuff 12 is decreased in width, a blood pressure value can be measured with accuracy.

The blood pressure monitor having the bag-shaped structure 122 included in the cuff is not necessarily a blood pressure monitor for a wrist. For example, the blood pressure monitor having the bag-shaped structure 122 included in the cuff may be a blood pressure monitor for an upper arm.

The blood pressure monitor having the aforementioned bag-shaped structure 122 included in the cuff may supply air to the bag-shaped structure 122 via a manual pump. Furthermore, instead of determining a blood pressure value based on a change in pulse wave detected by the pressure sensor, the blood pressure monitor having the aforementioned bag-shaped structure 122 included in the cuff may determine a blood pressure value based on a change in Korotkoff sound detected by a microphone or a stethoscope. In addition, instead of using the pressure sensor, the blood pressure monitor having the aforementioned bag-shaped structure 122 included in the cuff may use a mercury pressure gauge.

The bag-shaped structure 122 is not necessarily for use in a cuff of a blood pressure monitor. That is, the bag-shaped structure 122 may be used for other purposes. The technique described here is advantageously usable as long as it is used for the purpose for which the sheets constituting the bag-shaped structure 122 are desired to include a plurality of regions varied in at least one of flexibility and strength.

### EXAMPLES

Specific examples of the present invention are described below.

<Manufacture of Bag-Shaped Structure>

The bag-shaped structures 122 described with reference to FIGS. 3 and 4 were manufactured by the following methods.

### Example 1

First, a sheet made of a thermoplastic elastomer was manufactured. As this thermoplastic elastomer, a thermoplastic polyurethane resin (TPU) containing a trace amount of layered clay compound was used. The thickness of this sheet was 0.3 mm.

Next, this sheet was subjected to a local heat treatment using a semiconductor laser. Specifically, the sheet was heated at an irradiation position of a laser beam by repeatedly scanning a laser beam on the sheet in a pattern of lines with the focal diameter of 2 mm. The laser beam irradiation was performed on regions of this sheet that correspond to the bonded regions of the sheets 122a and 122b and their adjacent regions. The laser output was 0.3 W and the scanning speed was 20 mm/sec. This is a condition for a laser beam irradiation portion to reach a temperature of 70° C. to 120° C.

This sheet was then cut to obtain the sheets 122a and 122b. Thereafter, these sheets 122a and 122b were superimposed and bonded together at their peripheral portions.

In this manner, the bag-shaped structures 122 were manufactured.

### Example 2

The bag-shaped structures 122 described with reference to FIGS. 3 and 4 were manufactured by the same method as in Example 1 except that an infrared heater was used instead of the semiconductor laser for the heat treatment. As the infrared heater, a far infrared tube heater was used. The heat treatment with the far infrared tube heater was performed in a manner so that the heated portion reached a temperature of 70° C. to 120° C.

### Example 3

The bag-shaped structures 122 described with reference to FIGS. 3 and 4 were manufactured by the same method as in Example 1 except that a metal jig was used instead of the semiconductor laser for the heat treatment. Specifically, a cartridge heater was attached to the metal jig, and a portion to be heated of the sheet was nipped and heated with the metal jig. The heat treatment using this metal jig was performed in a manner so that the heated portion reached a temperature of 70° C. to 120° C.

### Comparative Example 1

Bag-shaped structures were manufactured by the same method as in Example 1 except that the heat treatment was omitted.

### Comparative Example 2

Bag-shaped structures were manufactured by the same method as in Example 3 except that the heat treatment using the metal jig was performed over the whole sheet.

<Measurement and Evaluation>

With respect to the bag-shaped structures obtained by the methods described above, a 100% Modulus was measured, while abnormal inflation and conformability to a living body were evaluated.

(Measurement of 100% Modulus)

For each of Examples 1 to 3 and Comparative Examples 1 and 2, a test piece for measuring a 100% Modulus was cut out from one of the bag-shaped structure structures. The test piece was set to “type 3 dumb-bell” specified in JIS 6251:2010. A 100% Modulus was measured according to the method specified in JIS K 6251:2010 (“Rubber, vulcanized or thermoplastic—Determination of tensile stress-strain properties”).

(Evaluation of Abnormal Inflation)

For each of Examples 1 to 3 and Comparative Examples 1 and 2, a cuff was manufactured using the bag-shaped structure obtained in each of these examples, and this cuff was fitted on a wrist. In this state, the bag-shaped structure was inflated by supplying compressed air thereto. The pressure of compressed air was 300 mmHg (=300×101325/760 Pa). The presence or absence of abnormal inflation was visually confirmed. This test was repeated three times, and the bag-shaped structure which did not cause abnormal inflation even once was evaluated as “o”, whereas the bag-shaped structure which caused abnormal inflation once or more was evaluated as “x”.

(Evaluation of Conformability to Living Body)

For each of Examples 1 to 3 and Comparative Examples 1 and 2, a cuff was prepared using the bag-shaped structure obtained in each of these examples. Next, a wrist blood pressure monitor was manufactured using each cuff, and a blood pressure value was measured by the wrist blood pressure monitor being fitted on a wrist. While the bag-shaped structure was inflated, the conformability to a wrist was examined. This test was also repeated three times, and the bag-shaped structure which exhibited good conformability to a wrist in all the tests was evaluated as “o”, whereas the bag-shaped structure which exhibited poor conformability in at least one test was evaluated as “x”.

The measurement result of a 100% Modulus and the evaluation result of abnormal inflation and conformability to a living body are shown in Table 1.

TABLE 1

| | | | | Comparative | Comparative | |
|---|---|---|---|---|---|---|
| | Example 1 | Example 2 | Example 3 | Example 1 | example 2 | |
| Heat treatment method | Laser | Infrared heater | Metal jig | None | Infrared heater | |
| Region not treated | 1.6 | 1.6 | 1.6 | 1.6 | — | 100% Modulus |
| with heat | | | | | | (MPa) |
| Region treated | 2.4 | 2.3 | 2.5 | — | 2.7 | |
| with heat | | | | | | |
| Pass number/ | 3/3 | 3/3 | 3/3 | 0/3 | 3/3 | Abnormal |
| Test number | | | | | | inflation |
| Determination | ∘ | ∘ | ∘ | x | ∘ | |
| Pass number/ | 3/3 | 3/3 | 3/3 | 3/3 | 0/3 | Conformability |
| Test number | | | | | | to living body |
| Determination | ∘ | ∘ | ∘ | ∘ | x | |

As shown in Table 1, each of the bag-shaped structures according to Examples 1 to 3 exhibits a sufficiently small 100% Modulus in the region to which the heat treatment was not performed, and a sufficiently large 100% Modulus in the region to which the heat treatment was performed. That is, it was found that these bag-shaped structures have high strength in a region in which abnormal inflation tends to occur while having sufficient flexibility in a region which comes in contact with a skin in the case of the bag-shaped structures being used in cuffs. In fact, when these bag-shaped structures were used in cuffs, no abnormal inflation occurred and the conformability to a living body was good. When a blood pressure value was measured using these bag-shaped structures in cuffs, the highly accurate measurement of a blood pressure value was achieved.

In Examples 1 to 3, as shown in FIG. 9, the heat treatment was performed to the regions of the sheets 122a and 122b that correspond to the bonded regions thereof and their adjacent regions. Assume that the whole sheet 122b is fixed to the cover member 121 or the curler 123. In this case, when compressed air is supplied to the bag-shaped structure 122, a portion in which the pressure is concentrated corresponds to a region of the sheet 122a that is located between the end of the region coming in contact with the wrist 2 and the bonded region of the sheet 122a. Therefore, abnormal inflation can be prevented by performing the heat treatment to at least this particular region.

In contrast, in the bag-shaped structure according to Comparative Example 1, a 100% Modulus was small over the entire sheet. When this bag-shaped structure was used in a cuff, abnormal inflation occurred. When a blood pressure value was measured using this bag-shaped structure in the cuff, a pressure was not applied to a living body effectively enough to measure a blood pressure value with high accuracy.

In the bag-shaped structure according to Comparative Example 2, a 100% Modulus was large over the entire sheet. When this bag-shaped structure was used in a cuff, abnormal inflation did not occur. However, in this bag-shaped structure, a region that comes in contact with a skin in the case of using the bag-shaped structure in a cuff lacked flexibility necessary for accurate measurement of a blood pressure value. When a blood pressure value was measured using this bag-shaped structure in the cuff, the conformability to a living body was not sufficient to measure a blood pressure value with high accuracy.

1. A bag-shaped structure comprising one or more sheets that contain a thermoplastic elastomer and are uniform in composition, wherein the one or more sheets comprise a plurality of regions that are different in 100% Modulus.

2. The bag-shaped structure according to claim 1, wherein the plurality of regions comprise a first region and a second region that is thinner and is higher in 100% Modulus than the first region.

3. The bag-shaped structure according to claim 1, wherein the one or more sheets are provided with one or more openings, the one or more sheets comprise a third region spaced apart from the one or more openings and a fourth region interposed between the one or more openings and the third region, and the fourth region has a 100% Modulus higher than that of the third region.

4. The bag-shaped structure according to claim 1, wherein the one or more sheets are provided with a plurality of bonded regions which are bonded to each other, and the one or more sheets comprise a fifth region comprising the plurality of bonded regions and a sixth region, at least part of the fifth region having a 100% Modulus higher than that of the sixth region.

5. The bag-shaped structure according to claim 1, wherein the one or more sheets comprise a seventh region, an eighth region that faces the seventh region in a case where the seventh region is applied on a living body, and a ninth region that connects an end of the seventh region and an end of the eighth region together, at least part of the ninth region having a 100% Modulus of higher than that of the seventh region.

6. The bag-shaped structure according to claim 5, wherein the ninth region includes a bonded region at which the one or more sheets are bonded to each other, and comprises a region whose 100% Modulus is higher than the seventh region between the bonded region of the ninth region and the seventh region.

7. A cuff for a blood pressure monitor, comprising the bag-shaped structure according to claim 1.

8. A blood pressure monitor comprising the cuff according to claim 7.

9. A method of manufacturing a bag-shaped structure, comprising heating a region of one or more sheets that contain a thermoplastic elastomer and are uniform in composition, to a temperature that is equal to or higher than a crystallization temperature of the thermoplastic elastomer and is lower than a melting point of the thermoplastic elastomer to increase a 100% Modulus at the region relative to another region.

10. The method of manufacturing a bag-shaped structure according to claim 9, wherein the one or more sheets further contain a nucleating agent.
